SEQUENCE LISTING (226 sequences)

SEQ ID NO: 1 - protein, 297 aa, Human respiratory syncytial virus A
  REGION 1..297: note = RSV G A strain Native
MSKNKDQRTAKTLERTWDTLNHLLFISSCLYKLNLKSVAQITLSILAMIISTSLIIVAIIFIASANHKITSTTTIIQDATNQIKNTTPTYLTQNPQLGISPSNPSDITSLITTILDSTTPGVKSTLQSTTVGTKNTTTTQAQPNKPTTKQRQNKPPSKPNNDFHFEVFNFVPCSICSNNPTCWAICKRIPNKKPGKRTTTKPTKKPTPKTTKKGPKPQTTKSKEAPTTKPTEEPTINTTKTNIITTLLTSNTTRNPELTSQMETFHSTSSEGNPSPSQVSITSEYPSQPSSPPNTPR

SEQ ID NO: 2 - protein, 233 aa, Human respiratory syncytial virus A
  REGION 1..233: note = RSV G ectodomain, residues 66-297
NHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKPTEEPTINTTKTNIITTLLTNNTTGNPKLTSQMETFHSTSSEGNLSPSQVSTTSEHPSQPSSPPNTTRQ

SEQ ID NO: 3 - protein, 43 aa, Human respiratory syncytial virus A
  REGION 1..43: note = RSV Gcc (central conserved region) A2 strain res 151-193. As described in Murata et al. (2010) Vaccine 28:6242-6246 at Fig. 1.
RQNKPPNKPNNDFHFEVFNFVPCSICSNNPTCWAICKRIPNKK

SEQ ID NO: 4 - protein, 180 aa, synthetic construct
  REGION 1..180: note = Synthetic: RSV Gcc B1-A2-foldon strain chimera sequence
MEESEESGGRKNPPKKPKDDYHFEVFNFVPCSICGNNQLCKSICKTIPNKKEEEEESEESGGRQNKPPSKPNNDFHFEVFNFVPCSICSNNPTCWAICKRIPNKKEEESSGGSGGGGSGGGGSGGGGSGSSAIGGYIPEAPRDGQAYVRKDGEWVLLSTFLGSGLEVLFQGPLEHHHHHH

SEQ ID NO: 5 - protein, 169 aa, synthetic construct
  REGION 1..169: note = Synthetic: RSV Gcc tetramer, A2-B1-A2-B1 strain chimera
MRQNKPPSKPNNDFHFEVFNFVPCSICSNNPTCWAICKRIPNKRKNPPKKPKDDYHFEVFNFVPCSICGNNQLCKSICKTIPNKKRQNKPPSKPNNDFHFEVFNFVPCSICSNNPTCWAICKRIPNKRKNPPKKPKDDYHFEVFNFVPCSICGNNQLCKSICKTIPNKK

SEQ ID NO: 6 - protein, 213 aa, synthetic construct
  REGION 1..213: note = Synthetic: RSV Gcc A2-alpha-B1 chimera particle
MEESEESGGRQNKPPSKPNNDFHFEVFNFVPCSICSNNPTCWAICKRIPNKEEEEESEESGGSGSWEEWNAKWDEWRNDQNDWREDWQAWRDDWAYWTLTWRYGELYSRLARIERRVEELRRLLQLIRHENRMVLQFVRALSMQARRLESKLEEEEESEESGGRKNPPKKPKDDYHFEVFNFVPCSICGNNQLCKSICKTIPNKKEESEESGG

SEQ ID NO: 7 - protein, 181 aa, synthetic construct
  REGION 1..181: note = Synthetic: RSV Gcc A2-A2-foldon strain chimera sequence
MEESEESGGRQNKPPSKPNNDFHFEVFNFVPCSICSNNPTCWAICKRIPNKKEEEEESEESGGRQNKPPSKPNNDFHFEVFNFVPCSICSNNPTCWAICKRIPNKKEEESSGGSGGGGSGGGGSGGGGSGSSAIGGYIPEAPRDGQAYVRKDGEWVLLSTFLGSGLEVLFQGPLEHHHHHH

SEQ ID NO: 8 - protein, 179 aa, synthetic construct
  REGION 1..179: note = Synthetic: RSV Gcc B1-B1-foldon strain chimera sequence
MEESEESGGRKNPPKKPKDDYHFEVFNFVPCSICGNNQLCKSICKTIPNKKEEEEESEESGGRKNPPKKPKDDYHFEVFNFVPCSICGNNQLCKSICKTIPNKKEEESSGGSGGGGSGGGGSGGGGSGSSAIGGYIPEAPRDGQAYVRKDGEWVLLSTFLGSGLEVLFQGPLEHHHHHH

SEQ ID NO: 9 - protein, 74 aa, synthetic construct
  REGION 1..74: note = Synthetic: alpha- sequence
DEWRNDQNDWREDWQAWRDDWAYWTLTWRYGELYSRLARIERRVEELRRLLQLIRHENRMVLQFVRALSMQARR

SEQ ID NO: 10 - protein, 40 aa, Human respiratory syncytial virus B
  REGION 1..40: note = RSV Gcc (central conserved region) B1 strain res 151-193. As described in Murata et al. (2010) Vaccine 28:6242-6246 at Fig. 1.
RLKNPPKKPKDDYHFEVFNFVPCSICGNNQLCKSICKTIP

SEQ ID NO: 11 - protein, 43 aa, synthetic construct
  REGION 1..43: note = Synthetic: Foldon
AIGGYIPEAPRDGQAYVRKDGEWVLLSTFLGSGLEVLFQGPLE

SEQ ID NO: 12 - protein, 60 aa, synthetic construct
  MOD_RES 1: note = Azido-PEG4 at 5' end
  REGION 1..60: note = Synthetic: RSV G peptide A2 (aa 151-193) for conjugation with N-terminal Azido linker and flanking glutamates
SGGSSGSSEEEGGSRQNKPPNKPNNDFHFEVFNFVPCSICSNNPTCWAICKRIPNKKEEE

SEQ ID NO: 13 - protein, 30 aa, synthetic construct
  REGION 1..30: note = Synthetic: 30 amino acid foldon
AIGGYIPEAPRDGQAYVRKDGEWVLLSTFL

SEQ ID NO: 14 - protein, 161 aa, synthetic construct
  REGION 1..161: note = Synthetic: RSV Gcc B1-A2-30 aa foldon strain chimera sequence
MEESEESGGRKNPPKKPKDDYHFEVFNFVPCSICGNNQLCKSICKTIPNKKEEEEESEESGGRQNKPPSKPNNDFHFEVFNFVPCSICSNNPTCWAICKRIPNKKEEESSGGSGGGGSGGGGSGGGGSGSSAIGGYIPEAPRDGQAYVRKDGEWVLLSTFL

SEQ ID NO: 15 - protein, 4 aa, synthetic construct
  REGION 1..4: note = Synthetic: linker
GGGS

SEQ ID NO: 16 - protein, 8 aa, synthetic construct
  REGION 1..8: note = Synthetic: linker
GGGSGGGS

SEQ ID NO: 17 - protein, 20 aa, synthetic construct
  REGION 1..20: note = Synthetic: linker
GGGSGGGSGGGSGGGSGGGS

SEQ ID NO: 18-200 - skipped (183 entries)

SEQ ID NO: 201 - protein, 173 aa, synthetic construct
  REGION 1..173: note = Synthetic: bfpFerritin-N19Q/C31S/S26C
ESQVRQQFSKDIEKLLNEQVNKEMQSSNLYMCMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 202 - protein, 173 aa, synthetic construct
  REGION 1..173: note = Synthetic: bfpFerritin-N19Q/C31S/S72C
ESQVRQQFSKDIEKLLNEQVNKEMQSSNLYMSMSSWSYTHSLDGAGLFLFDHAAEEYEHAKKLIIFLNENNVPVQLTCISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 203 - protein, 173 aa, synthetic construct
  REGION 1..173: note = Synthetic: bfpFerritin-N19Q/C31S/A75C
ESQVRQQFSKDIEKLLNEQVNKEMQSSNLYMSMSSWSYTHSLDGAGLFLFDHAAEEYEHAKKLIIFLNENNVPVQLTSISCPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 204 - protein, 173 aa, synthetic construct
  REGION 1..173: note = Synthetic: bfpFerritin- N19Q/C31S/K79C
ESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 205 - protein, 173 aa, synthetic construct
  REGION 1..173: note = Synthetic: bfpFerritin-N19Q/C31S/S100C
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISECINNIVDHAIKSKDHATFNFLQWYVAEQHEEEVLFKDILDKIELIGNENHGLYLADQYVKGIAKSRKS

SEQ ID NO: 206 - protein, 173 aa, synthetic construct
  REGION 1..173: note = Synthetic: bfpFerritin-N19Q/C31S/S111C
ESQVRQQFSKDIEKLLNEQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKS

SEQ ID NO: 207 - protein, 173 aa, synthetic construct
  REGION 1..173: note = Synthetic: bfpFerritin-N19Q/C31S/E12C
ESQVRQQFSKDIEKLLNCQVNKEMQSSNLYMSMSSWSYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 208 - protein, 173 aa, Helicobacter pylori
  REGION 1..173: note = Exemplary H. pylori J99 Ferritin with bullfrog linker
ESQVRQQFSKDIEKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 209 - protein, 166 aa, Helicobacter pylori
  REGION 1..166: note = Exemplary wild-type H. pylori J99 ferritin (without bullfrog linker or N-terminal Met)
LSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 210 - DNA, 22 bp, synthetic construct
  misc_feature 1..22: note = Synthetic: CpG (ISS-1018)
  source 1..22: mol_type = other DNA
tgactgtgaacgttcgagatga

SEQ ID NO: 211 - protein, 191 aa, Trichoplusia ni
  REGION 1..191: note = Trichoplusia ni heavy chain ferritin
TQCNVNPVQIPKDWITMHRSCRNSMRQQIQMEVGASLQYLAMGAHFSKDVVNRPGFAQLFFDAASEEREHAMKLIEYLLMRGELTNDVSSLLQVRPPTRSSWKGGVEALEHALSMESDVTKSIRNVIKACEDDSEFNDYHLVDYLTGDFLEEQYKGQRDLAGKASTLKKLMDRHEALGEFIFDKKLLGIDV

SEQ ID NO: 212 - protein, 212 aa, Trichoplusia ni
  REGION 1..212: note = Trichoplusia ni light chain ferritin
ADTCYNDVALDCGITSNSLALPRCNAVYGEYGSHGNVATELQAYAKLHLERSYDYLLSAAYFNNYQTNRAGFSKLFKKLSDEAWSKTIDIIKHVTKRGDKMNFDQHSTMKTERKNYTAENHELEALAKALDTQKELAERAFYIHREATRNSQHLHDPEIAQYLEEEFIEDHAEKIRTLAGHTSDLKKFITANNGHDLSLALYVFDEYLQKTV

SEQ ID NO: 213 - protein, 174 aa, Pyrococcus furiosus
  REGION 1..174: note = Pyrococcus furiosus ferritin
MLSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPGLLMQGGE

SEQ ID NO: 214 - protein, 183 aa, Homo sapiens
  REGION 1..183: note = human heavy chain ferritin
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVQQSLLELHKLATDKNDPHLCDFIETHYLNEQVKAIKELGDHVTNLRKMGAPESGLAEYLFDKHTLGDSDQES

SEQ ID NO: 215 - protein, 204 aa, Homo sapiens
  REGION 1..204: note = human light chain ferritin
MDSKGSSQKGSRLLLLLVVSNLLLPQGVLASSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 216 - protein, 154 aa, Aquifex aeolicus
  REGION 1..154: note = lumazine synthase from Aquifex aeolicus
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 217 - protein, 8 aa, unidentified
  REGION 1..8: note = Bullfrog linker
ESQVRQQF

SEQ ID NO: 218 - protein, 15 aa, synthetic construct
  REGION 1..15: note = Synthetic: Cysteine-Thrombin-His Linker
CLVPRGSLEHHHHHH

SEQ ID NO: 219 - protein, 156 aa, synthetic construct
  REGION 1..156: note = Synthetic: E. coli 6,7-dimethyl-8-ribityllumazine synthase
MNIIEANVATPDARVAITIARFNNFINDSLLEGAIDALKRIGQVKDENITVVWVPGAYELPLAAGALAKTGKYDAVIALGTVIRGGTAHFEYVAGGASNGLAHVAQDSEIPVAFGVLTTESIEQAIERAGTKAGNKGAEAALTALEMINVLKAIKA

SEQ ID NO: 220 - protein, 16 aa, synthetic construct
  REGION 1..16: note = Synthetic: 16 amino acid linker
GGGGSGGGGSGGGGSG

SEQ ID NO: 221 - protein, 28 aa, synthetic construct
  REGION 1..28: note = Synthetic: 28 amino acid linker
GGSGSGSNSSASSGASSGGASGGSGGSG

SEQ ID NO: 222 - protein, 46 aa, synthetic construct
  REGION 1..46: note = Synthetic: 46 amino acid linker
GGSGSASSGASASGSSNGSGSGSGSNSSASSGASSGGASGGSGGSG

SEQ ID NO: 223 - protein, 26 aa, synthetic construct
  REGION 1..26: note = Synthetic: FR1
GGSGSASAEAAAKEAAAKAGGSGGSG

SEQ ID NO: 224 - protein, 32 aa, synthetic construct
  REGION 1..32: note = Synthetic: FR2
GGSGSASAEAAAKEAAAKEAAAKASGGSGGSG

SEQ ID NO: 225 - skipped

SEQ ID NO: 226 - protein, 299 aa, Human respiratory syncytial virus B
  REGION 1..299: note = RSV G B strain Native
MSKHKNQRTARTLEKTWDTLNHLIVISSCLYRLNLKSIAQIALSVLAMIISTSLIIAAIIFIISANHKVTLTTVTVQTIKNHTEKNITTYLTQVPPERVSSSKQPTTTSPIHTNSATTSPNTKSETHHTTAQTKGRTTTSTQTNKPSTKPRLKNPPKKPKDDYHFEVFNFVPCSICGNNQLCKSICKTIPSNKPKKKPTIKPTNKPTTKTTNKRDPKTPAKTTKKETTTNPTKKPTLTTTERDTSTSQSTVLDTTTLEHTIQQQSLHSTTPENTPNSTQTPTASEPSTSNSTQNTQSHA

(19) United States
(12) Patent Application Publication
(10) Pub. No.: US 2026/0265309 A1
(43) Pub. Date: Sep. 10, 2026

(54) Antigenic Multimeric Respiratory Syncytial Virus Polypeptides

(71) Applicant: Sanofi, Paris (FR)

(72) Inventors: Kurt Swanson, Bridgewater, NJ (US); Chih-Jen Wei, Bridgewater, NJ (US); Gary J. Nabel, Bridgewater, NJ (US)

(73) Assignee: Sanofi, Paris (FR)

(21) Appl. No.: 19/345,398

(22) Filed: Sep. 30, 2025

Related U.S. Application Data
(63) Continuation of application No. 17/489,249, filed on Sep. 29, 2021, now Pat. No. 12,454,553.
Application No. 17/489,249 is a continuation of application No. PCT/US2020/026198, filed on Apr. 1, 2020.
(60) Provisional application No. 62/828,302, filed on Apr. 2, 2019.

(51) Int. Cl. C07K 14/005; A61K 39/00; A61K 39/155; A61P 31/14; C12N 7/00

(52) U.S. Cl. CPC C07K 14/005; A61K 39/155; A61P 31/14; C12N 7/00; A61K 2039/64; A61K 2039/70; C12N 2760/18522; C12N 2760/18534; C12N 2760/18571

(57) ABSTRACT

This disclosure relates to antigenic multimeric respiratory syncytial virus (RSV) G polypeptides for use in eliciting an immune response to RSV.

This application is a continuation of U.S. patent application Ser. No. 17/489,249, filed Sep. 29, 2021, which is a continuation of International Application No. PCT/US2020/026198, filed on Apr. 1, 2020, which claims the benefit of priority to U.S. Provisional Application No. 62/828,302, filed on Apr. 2, 2019, each of which are incorporated by reference in their entirety.

The instant application contains a Sequence Listing which has been submitted electronically in XML format and is hereby incorporated by reference in its entirety. Said XML copy, created on Sep. 25, 2025, is named 01121-0042-01US-ST26.xml and is 95,953 bytes in size.

Even with many successes in the field of vaccinology, new breakthroughs are needed to protect humans against many life-threatening infectious diseases. Many currently licensed vaccines rely on decade-old technologies to produce live-attenuated or inactivated killed pathogens, which carry inherent safety concerns and in many cases, stimulate only short-lived, weak immune responses that require the administration of multiple doses. While advances in genetic and biochemical engineering have made it possible to develop therapeutic agents to challenging disease targets, these applications to the field of vaccinology have not been fully realized. Recombinant polypeptide technologies now allow the design of optimal antigens. Additionally, nanoparticles have increasingly demonstrated the potential for optimal antigen presentation and targeted drug delivery. Nanoparticles with multiple attached antigens have been shown to have increased binding avidity afforded by the multivalent display of their molecular cargos, and an ability to cross biological barriers more efficiently due to their nanoscopic size. Helicobacter pylori (H. pylori) ferritin nanoparticles fused to influenza virus haemagglutinin (HA) polypeptide has allowed improved antigen stability and increased immunogenicity in mouse influenza models (see Kanekiyo et al., Nature 499:102-106 (2013)). This fusion polypeptide self-assembled into an octahedrally-symmetric nanoparticle and presented 8 trimeric HA spikes to give a robust immune response in various pre-clinical models when used with an adjuvant.

Respiratory syncytial virus (RSV) is a leading cause of severe respiratory disease in infants and a major cause of respiratory illness in the elderly. It remains an unmet vaccine need despite decades of research. While the need for a vaccine is clear, development of an RSV vaccine was stymied in the 1960's when a clinical trial using a formalin inactivated RSV virus made disease, following RSV infection, more severe in infants. See, Hurwitz (2011) Expert Rev Vaccines 10(10): 1415-1433. More recently, clinical programs using an RSV F antigen in its post-fusion conformation failed to elicit sufficient efficacy in adults. See, Faloon et al. (2017) JID 216:1362-1370.

RSV G is a largely unstructured polypeptide with the first and last third of the molecule comprising several O-glycosylation sites. The ectodomain of RSV G, when expressed in mammalian cells, is by mass more glycan than amino acid. These flanking regions are also poorly conserved, giving rise to the name the G hypervariable regions. In contrast, the central region of G is fairly well conserved between the two major strains of RSV, the A and the B strain. Thus, this domain is referred to as the RSV G central conserved region (Gcc).

The Gcc region is further subdivided into the proximal region, a structured loop stabilized by hydrophobic and proline-rich sequences, and the distal region, consisting of two helices held together by two disulfide bonds forming the so-called “cystine noose.” The distal cysteine noose contains the CX3C motif demonstrated to be the binding motif of the virus for the CX3C receptor on the HAE cells. The proximal region harbors the only well-characterized neutralizing epitope for RSV G, the 131-2G epitope. While no structure exists for the complete RSV Gcc, data suggests the two structured regions form a higher tertiary structure which places the proximal region in close proximity to the distal region, thus explaining why the NAb 131-2G can sterically block the CX3C site and thus inhibit binding of the virus to the CX3CR receptor.

The Gcc alone is a poor immunogen, given the small size of the approximately 30 amino acid domain. We previously demonstrated that this domain can be synthesized as a peptide and chemically conjugated to the ferritin nanoparticle forming the bio-conjugate Gcc-NP. This lead antigen elicits a potent neutralizing response as observed in the HAE neutralizing assay. Furthermore, the antigen lacks the potentially immune distracting hypervariable regions which would make the G ectomain a poor vaccine candidate.

Here, a set of new polypeptides, nanoparticles, compositions, methods, and uses involving RSV G polypeptides is presented. Novel RSV G polypeptides were generated, including polypeptides in which the Gcc peptide is presented as a multimeric antigen. Gcc polypeptides from both an RSV A strain (the laboratory A2 strain) and an RSV B strain (the laboratory B1 strain) were utilized to provide broader scope of protection.

### SUMMARY

It is an object of this disclosure to provide compositions, kits, methods, and uses that can provide one or more of the advantages discussed above, or at least provide the public with a useful choice.

In some embodiments, an antigenic RSV G polypeptide is provided comprising more than two RSV Gcc monomers, wherein the polypeptide does not comprise ferritin. In some embodiments, the antigenic RSV G polypeptide is a single chain.

In some embodiments, antigenic RSV G polypeptides are provided, wherein the G polypeptide is a Gcc polypeptide displayed as a particle (e.g., multimer). In some embodiments, a dimer comprising one Gcc A strain and on Gcc B strain are fused together genetically with an N- or C-terminal foldon tag. Upon refolding from the cell in which is it expressed, e.g., E. coli, the foldon tag trimerizes, and the resulting particle presents six copies of the Gcc polypeptide (3 of A and 3 of B strain), which is sometimes referred to herein as “Gcc B1-A2-foldon” or “Gcc hexamer.” In some embodiments, four Gcc peptides (in any order, including from N- to C-terminus B1, A2, B1 and A2) are genetically fused together with glutamate rich linkers to produce a soluble tetramer of Gcc peptides (sometimes herein referred to herein as “Gcc Tetramer”). The Gcc tetramer lacks a multimerization domain and therefore does not form a particle.

Further embodiments are disclosed herein as follows:
Embodiment 01. An antigenic RSV G polypeptide comprising more than two RSV Gcc monomers, wherein the polypeptide does not comprise ferritin.
Embodiment 02. The antigenic RSV G polypeptide of embodiment 1, wherein the polypeptide is a single chain.
Embodiment 03. An antigenic RSV G single chain polypeptide comprising more than two RSV Gcc monomers.
Embodiment 04. The antigenic RSV G polypeptide of any one of the preceding embodiments, comprising 3, 4, 5, 6, 7, 8, 9, or 10 Gcc monomers.
Embodiment 05. The antigenic RSV G polypeptide of any one of the preceding embodiments, comprising a) Gcc monomers of strain A only; b) Gcc monomers of strain B only; or c) Gcc monomers of strain A and strain B.
Embodiment 06. The antigenic RSV G polypeptide of any one of the preceding embodiments, comprising more than two Gcc monomers of strain A.
Embodiment 07. The antigenic RSV G polypeptide of any one of the preceding embodiments, comprising more than two Gcc monomers of strain B.
Embodiment 08. The antigenic RSV G polypeptide of any one of the preceding embodiments, comprising at least one Gcc monomer of strain A and at least one Gcc monomer of strain B.
Embodiment 09. The antigenic RSV G polypeptide of any one of the preceding embodiments, wherein the polypeptide is a trimer, tetramer, or hexamer.
Embodiment 10. The antigenic RSV G polypeptide of any one of the preceding embodiments, comprising three Gcc monomers of strain A.
Embodiment 11. The antigenic RSV G polypeptide of any one of the preceding embodiments, comprising three Gcc monomers of strain B.
Embodiment 12. The antigenic RSV G polypeptide of any one of the preceding embodiments, comprising three Gcc monomers of strain A and three Gcc monomers of strain B.
Embodiment 13. The antigenic RSV G polypeptide of any one of the preceding embodiments, comprising a multimerization domain.
Embodiment 14. The antigenic RSV G polypeptide of any one of the preceding embodiments, comprising 1, 2, 3, 4, 5, 6, 7, 8, 9, or 10 multimerization domains.
Embodiment 15. The antigenic RSV G polypeptide of embodiment 13 or 14, wherein the multimerization domain is a foldon.
Embodiment 16. The antigenic RSV G polypeptide of embodiment 13 or 14, wherein the multimerization domain comprises SEQ ID NO: 9.
Embodiment 16.1. The antigenic RSV G polypeptide of embodiment 13 or 14, wherein the multimerization domain comprises SEQ ID NO: 13.
Embodiment 17. The antigenic RSV G polypeptide of any one of the preceding embodiments, comprising two Gcc monomers of strain A and two Gcc monomer of strain B.
Embodiment 18. The antigenic RSV G polypeptide of embodiment 17, wherein the monomers of strain A and B alternate in order in a single chain.
Embodiment 19. The antigenic RSV G polypeptide of any one of the preceding embodiments, comprising an amino acid sequence with 80%, 85%, 90%, 95%, 98%, 99%, or 100% identity to any one of SEQ ID NOs: 4-8.
Embodiment 20. The antigenic RSV G polypeptide of any one of the preceding embodiments, wherein the antigenic RSV G polypeptide is encoded by a polynucleotide encoding at least two RSV Gcc monomers and at least one multimerization domain; or at least three RSV Gcc monomers.
Embodiment 21. The antigenic RSV G polypeptide of embodiment 20, wherein the antigenic RSV G polypeptide is encoded by a polynucleotide encoding at least two RSV Gcc monomers and at least one multimerization domain.
Embodiment 22. The antigenic RSV G polypeptide of embodiment 21, wherein at least one Gcc monomer is from strain A and at least one Gcc monomer is from strain B.
Embodiment 23. The antigenic RSV G polypeptide of any one of embodiments 20-22, wherein the polynucleotide encoding the RSV Gcc from strain A encodes:
   a. the same sequence of amino acids as SEQ ID NO: 3; or
   b. the same sequence of amino acids as SEQ ID NO: 3 without the C-terminal K; or
   c. the same sequence of amino acids as amino acids 2-42 of SEQ ID NO: 4.
Embodiment 24. The antigenic RSV G polypeptide of any one of embodiments 20-22, wherein the polynucleotide encoding the RSV Gcc from strain B encodes:
   a. the same sequence of amino acids as SEQ ID NO: 10; or
   b. the same sequence of amino acids as amino acids 10-51 of SEQ ID NO: 8.
Embodiment 25. The antigenic RSV G polypeptide of any one of embodiments 20-24, wherein the polynucleotide encoding the multimerization domain encodes the same sequence of amino acids as:
   a. SEQ ID NO: 11; or
   b. amino acids 132-175 of SEQ ID NO: 7.
Embodiment 25.1. The antigenic RSV G polypeptide of any one of embodiments 13, 15, 16.1, 19, or 22, comprising an amino acid sequence with 80%, 85%, 90%, 95%, 98%, 99%, or 100% identity to SEQ ID NO: 14.
Embodiment 26. The antigenic RSV G polypeptide of any one of embodiments 20-25.1, capable of forming a Gcc particle upon expression of the polynucleotide in a host cell.
Embodiment 27. The antigenic RSV G polypeptide of embodiment 26, wherein the polypeptide is a particle and the particle is a hexamer.
Embodiment 28. The antigenic RSV G polypeptide of embodiment 26, wherein the hexamer comprises three RSV Gcc strain A monomers and three RSV Gcc strain B monomers.
Embodiment 29. The antigenic RSV G polypeptide of embodiment 20, wherein the antigenic RSV G polypeptide is encoded by a polynucleotide encoding at least three RSV Gcc monomers.
Embodiment 30. The antigenic RSV G polypeptide of embodiment 29, wherein the polynucleotide encodes four RSV Gcc monomers.
Embodiment 31. The antigenic RSV G polypeptide of embodiment 30, wherein the polynucleotide encodes two Gcc monomers are of strain A and two Gcc monomers are of strain B.
Embodiment 32. The antigenic RSV G polypeptide of embodiment 31, wherein the polynucleotide encodes the two Gcc monomers of strain A and the two Gcc monomers of strain B in alternating order.
Embodiment 33. The antigenic RSV G polypeptide of any one of the preceding embodiments, wherein the antigenic RSV G polypeptide is capable of eliciting an immune response to RSV and/or protecting a subject against RSV infection.
Embodiment 34. A composition comprising the antigenic RSV G polypeptide of any one of embodiments 1-33, further comprising a pharmaceutically acceptable carrier.
Embodiment 35. The antigenic RSV G polypeptide or composition of any one of embodiments 1-34 for use in a method of eliciting an immune response to RSV or in protecting a subject against RSV infection.
Embodiment 36. A method of eliciting an immune response to RSV or protecting a subject against RSV infection comprising administering the antigenic RSV G polypeptide or composition of any one of embodiments 1-34 to a subject.
Embodiment 37. The antigenic RSV G polypeptide or composition of embodiment 34, or the method of embodiment 36, wherein the subject is human.
Embodiment 38. A nucleic acid encoding the antigenic RSV polypeptide of any one of embodiments 1-33, optionally wherein the nucleic acid is an mRNA.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate several embodiments and together with the description, serve to explain the principles described herein.

### BRIEF DESCRIPTION OF THE DRAWINGS

FIGS. 1A-B. Characterization of RSV G central domain peptide (Gcc) conjugated to ferritin nanoparticle. (FIG. 1A) Coomassie-stained SDS-PAGE gel showing the click-conjugation of RSV G central domain (SEQ ID NO. 12) to ferritin nanoparticle, forming the Gcc-NP antigen. (FIG. 1B) Structural model of Gcc-NP.

FIGS. 2A-C. Exemplary RSV chimeric antigens. (FIG. 2A) One B1 strain Gcc peptide sequence is fused to an A2 strain Gcc peptide sequence followed by a foldon trimerization tag. A modeled structure of the hypothetical folded construct is displayed where the tandem Gcc peptides trimerize by virtue of the foldon tag and thus display 6 copies of the Gcc peptide. Sometimes referred to as “B1-A2-foldon.” (FIG. 2B) Two copies each of Gcc peptide sequence from A and B strain are fused in the (optionally in the order A2-B1-A2-B1) forming a tetramer chimera polypeptide. Unlike B1-A2-foldon, this construct does not require folding into a higher tertiary structure. (FIG. 2C) One copy of the A2 strain Gcc peptide is fused to an α particle protomer at its N-terminus while one copy of the B1 strain Gcc peptide is fused to the C-terminus. A modeled structure of the hypothetical folded construct is shown where the α protomers form a particle core with numerous copies of the Gcc peptides displayed on the surface.

FIGS. 3A-C. Neutralizing antibody titers elicited by a low dose (0.5 μg) of RSV G antigens. (FIG. 3A) RSV A strain HAE neutralizing titer elicited from immunization with G antigens formulated with AF03 from sera taken two weeks post the second immunization (2wp2). Neutralizing responses from naïve sera and a hyperimmune sera are shown as negative and positive controls, respectively. In each graph, immunogen used in the immunization is indicated below the x-axis. (FIG. 3B) RSV A strain HAE neutralizing titer elicited from immunization with G antigen formulated with AF03 from sera taken two weeks post the third immunization. Control sera and immunogens are labeled. (FIG. 3C) RSV B strain HAE neutralizing titer elicited from immunization with G antigen formulated with AF03 from sera taken two weeks post the third immunization. Control sera and immunogens are labeled.

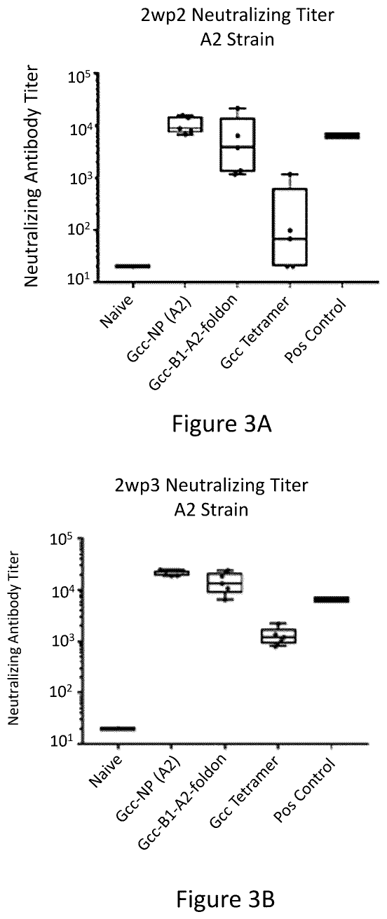

FIGS. 4A-B. RSV A2 strain antigen-binding antibody responses elicited by RSV G antigens. (FIG. 4A) Gcc-binding antibody responses elicited to the Gcc A2 strain measured at two weeks post the second injection (light grey boxes) and two weeks post the third injection (dark grey boxes) elicited by a high dose (5 μg) of RSV G chimeric antigens. Naïve mouse sera response is shown as a negative control. Immunogens are labeled below the x-axis. (FIG. 4B) Gcc-binding antibody responses elicited to the Gcc A2 strain measured at two weeks post the second injection (light grey boxes) and two weeks post the third injection (dark grey boxes) elicited by the low dose (0.5 μg) of RSV G chimeric antigens. Naïve mouse sera and immunogens are labeled as above.

FIGS. 5A-B. RSV B1 strain antigen-binding antibody responses elicited by RSV G antigens. (FIG. 5A) Gcc-binding antibody responses elicited to the Gcc B1 strain measured at two weeks post the second injection (light grey boxes) and two weeks post the third injection (dark grey boxes) elicited by a high dose (5 μg) of RSV G chimeric antigens. Naïve mouse sera response is shown as a negative control. Immunogens are labeled below the x-axis. (FIG. 5B) Gcc-binding antibody responses elicited to the Gcc B1 strain measured at two weeks post the second injection (light grey boxes) and two weeks post the third injection (dark grey boxes) elicited by a low dose (0.5 μg) of RSV G chimeric antigens. Naïve mouse sera and immunogens are labeled as above.

FIG. 6A is a schematic illustration of the antibody-binding experiment described in Example 5.

FIG. 6B shows results from the antibody-binding experiment described in Example 5 from the reference antigen (circles) and the construct of SEQ ID NO: 14 (squares). The dashed line indicates the baseline signal from blank wells. The units of the vertical and horizontal axes are Log OD units (UDO) and Log AU/mL, respectively (AU: arbitrary units).

### DETAILED DESCRIPTION

Antigenic RSV G polypeptides are provided that are antigenic when administered alone, or with adjuvant as a separate molecule. In some embodiments, the antigenic RSV G polypeptides comprise more than two RSV Gcc monomers. In some embodiments, the RSV G polypeptides do not comprise ferritin. In some embodiments, the RSV G polypeptides are single chain. In some embodiments, the RSV G polypeptides comprise a multimerization domain. In some embodiments, the RSV G polypeptides are particles/multimers, e.g., timers, tetramers, or hexamers.

### A. Definitions

“F polypeptide,” or “RSV F polypeptide” refers to the polypeptide of RSV responsible for driving fusion of the viral envelope with host cell membrane during viral entry.

“G polypeptide” or “RSV G polypeptide” as used herein, refers to the attachment polypeptide responsible for associating RSV with human airway epithelial cells. An exemplary wild-type RSV G amino acid sequence is provided as SEQ ID NO: 1. RSV G polypeptide comprises an ectodomain (approximately amino acids 66-297 of RSV G (SEQ ID NO: 2)) that resides extracellularly. Within the ectodomain of RSV G is a central conserved region (Gcc or CCR, approximately amino acids 151-193 of SEQ ID NO: 1). The CCR of RSV G comprises a CX3C motif. The CX3C motif mediates binding of G polypeptide to the CX3CR1 receptor.

“Protomer,” as used herein, refers to a structural unit of an oligomeric polypeptide.

“Ferritin” or “ferritin polypeptide,” as used herein, refers to a polypeptide with detectable sequence identity to H. pylori ferritin (SEQ ID NO: 208 or 209) or another ferritin, such as P. furiosus ferritin, Trichoplusia ni ferritin, or human ferritin, that serves to store iron, e.g., intracellularly or in tissues or to carry iron in the bloodstream. Such exemplary ferritins, include those that occur as two polypeptide chains, known as the heavy and light chains (e.g., T. ni and human ferritin). A ferritin may be a fragment of a full-length naturally-occurring sequence.

As used herein, a “monomer” refers to a single molecule that has not assembled with other molecules.

As used herein, “particle,” or “multimer” refers to a self-assembled globular form. Exemplary “particles” include constructs comprising a multimerization domain (e.g., foldon). A multimerization domain may function to brings multiple copies of a molecule together. “Particle” and “multimer” are used interchangeably herein except where differentiated. An exemplary particle is the “Gcc B1-A2-foldon,” which forms a hexamer (three copies of the Gcc B1 and three copies of the Gcc A2). The “Gcc tetramer” described herein is not a “particle/multimer” as it is not a self-assembled globular form.

An “RSV Gcc polypeptide” includes monomeric and particle/multimer forms of RSV Gcc.

“Immune response,” as used herein, refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a “protective immune response” refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. An “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens.

“Adjuvant,” as used herein, refers to a substance or vehicle that non-specifically enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules. An adjuvant may be administered as a separate molecule in a composition or covalently bound (conjugated) to a particle.

An “antigenic RSV G polypeptide” is used herein to refer to a polypeptide comprising all or part of an RSV G amino acid sequence of sufficient length that the molecule is antigenic with respect to RSV. Antigenicity may be a feature of the RSV sequence as part of a construct further comprising a heterologous sequence, such as an α sequence. That is, if an RSV sequence is part of a construct further comprising a heterologous sequence, then it is sufficient that the construct can serve as an antigen that generates anti-RSV antibodies, regardless of whether the RSV sequence without the heterologous sequence could do so.

An “α sequence” is the sequence of SEQ ID NO: 9.

In some embodiments, an “antigenic RSV G α polypeptide” comprises a polypeptide having at least 80%, 85%, 90%, 95%, 99%, or 100% identity to SEQ ID NO: 6. In some embodiments, the antigenic RSV G α polypeptide comprises a single chain polypeptide comprising an RSV Gcc and SEQ ID NO: 9, or a fragment of SEQ ID NO: 9 capable of multimerizing the RSV Gcc. The antigenic RSV G α polypeptide may further comprise an immune-stimulatory moiety. Antigenicity may be a feature of the RSV G sequence as part of the larger construct. That is, it is sufficient that the construct can serve as an antigen against the RSV G polypeptide, regardless of whether the RSV G polypeptide without the a (and immune-stimulatory moiety if applicable) could do so. To be clear, however, an antigenic RSV G polypeptide does not need to comprise an α sequence. “Antigenic RSV G polypeptide” is used herein to refer to a polypeptide which may be either an antigenic RSV G α polypeptide or an antigenic RSV G polypeptide that does not comprise an α sequence.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically-engineered animal, and/or a clone. In certain embodiments of the present invention the subject is an adult, an adolescent or an infant. In some embodiments, terms “individual” or “patient” are used and are intended to be interchangeable with “subject.”

As used herein, the term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

The disclosure describes sequences having a certain degree of identity to a given nucleic acid sequence or amino acid sequence, respectively (a references sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes the ability to act as a cytokine, in particular when administered to a subject. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### B. Antigenic RSV G Polypeptides

Provided herein are antigenic RSV G polypeptides comprising more than two RSV Gcc polypeptides. In some embodiments, the Gcc polypeptides are Gcc monomers. In some embodiments, antigenic RSV G polypeptides comprising more than two RSV Gcc monomers are provided, wherein the polypeptide does not comprise ferritin. In some embodiments, an antigenic RSV G polypeptide is provided as a single chain, wherein the single chain comprises more than two RSV Gcc monomers. The RSV Gcc polypeptide/monomer may comprise the whole sequence of RSV Gcc or a portion of RSV Gcc. In some embodiments, the RSV Gcc is lacking the last one, two, or three amino acids. In some embodiments, the RSV Gcc is lacking the last amino acid (e.g., the K at the N-terminus of SEQ ID NO: 3). The RSV Gcc polypeptide may comprise modifications compared to a wildtype sequence (SEQ ID NO: 3), such as, for example, an N→S substitution at amino acid number 7 of SEQ ID NO: 3.

In some embodiments, the RSV Gcc polypeptide is from RSV A strain (UniProtKB/Swiss-Prot: P27022.1; SEQ ID NO: 1). In some embodiments, the RSV Gcc polypeptide is from RSV B strain (UniProtKB/Swiss-Prot: 036633.1; SEQ ID NO: 226). In some embodiments, the RSV Gcc polypeptide from strain A comprises the amino acids of SEQ ID NO: 3. In some embodiments, the RSV Gcc polypeptide from strain A comprises the amino acids of SEQ ID NO: 3, wherein the terminal K is of SEQ ID NO: 3 is not present. In some embodiments, the RSV Gcc polypeptide from strain A comprises amino acids 2-42 of SEQ ID NO: 4. In some embodiments, the RSV Gcc polypeptide from strain B comprises the amino acids of SEQ ID NO: 10. In some embodiments, the RSV Gcc polypeptide from strain B comprises amino acids 10-51 of SEQ ID NO: 8.

In some embodiments, the RSV Gcc polypeptide comprises all or part of the Gcc region (amino acids 151-193 of RSV G (SEQ ID NO: 1)). In some embodiments, the RSV G polypeptide comprises a CX3C motif. In some embodiments, the RSV G polypeptide binds to the CX3CR1 receptor.

In some embodiments, the RSV G polypeptide is not glycosylated. For example, an RSV G polypeptide can lack NXS/TX glycosylation sites, either due to truncation or mutation of N or S/T residues (e.g., to Q or A, respectively), or a combination thereof.

In some embodiments, an RSV G polypeptide comprises 3, 4, 5, 6, 7, 8, 9, or 10 Gcc monomers. In some embodiments, an RSV G polypeptide comprises 1-2, 1-5, 1-10, 1-20, 1-25, 1-30, 1-40, 1-50, 1-60, 1-70, 1-80, 1-90, 1-100, or 1-120 Gcc monomers. In some embodiments, an RSV G polypeptide comprises 3 Gcc monomers. In some embodiments, an RSV G polypeptide comprises 6 Gcc monomers. In some embodiments, an RSV G polypeptide comprises 120 Gcc monomers. In some embodiments, the antigenic RSV G polypeptide comprises Gcc monomers of strain A only. In some embodiments, the antigenic RSV G polypeptide comprises Gcc monomers of strain B only. In some embodiments, the antigenic RSV G polypeptide comprises Gcc monomers of strain A and strain B.

In some embodiments, the antigenic RSV G polypeptide comprises more than two Gcc monomers of strain A. In some embodiments, the antigenic RSV G polypeptide comprises more than two Gcc monomers of strain B. In some embodiments, the antigenic RSV G polypeptide comprises at least one Gcc monomer of strain A and at least one Gcc monomer of strain B. In some embodiments, the antigenic RSV G polypeptide comprises one Gcc monomer of strain A and one Gcc monomer of strain B. In some embodiments, the antigenic RSV G polypeptide comprises two Gcc monomers, both from strain A. In some embodiments, the antigenic RSV G polypeptide comprises two Gcc monomers, both from strain B. In some embodiments, the antigenic RSV G polypeptide is a single chain.

In some embodiments, the antigenic RSV G polypeptide comprises three Gcc monomers of strain A. In some embodiments, the antigenic RSV G polypeptide comprises three Gcc monomers of strain B. In some embodiments, the antigenic RSV G polypeptide comprises three Gcc monomers of strain A and three Gcc monomers of strain B.

In some embodiments, the antigenic RSV G polypeptide is a trimer, tetramer, or hexamer. In some embodiments, the antigenic RSV G polypeptide is a tetramer. In some embodiments, the trimer, tetramer, or hexamer is a single chain polypeptide. In some embodiments, the trimer, tetramer, or hexamer is a single chain polypeptide that does not form a particle. In some embodiments, the antigenic RSV G polypeptide is a tetramer comprising four Gcc monomers. In some embodiments, the antigenic RSV G polypeptide is a tetramer comprising four Gcc monomers, wherein two monomers of strain A and two monomers of strain B. In some embodiments, the antigenic RSV G polypeptide is a tetramer comprising four Gcc monomers, wherein two monomers of strain A and two monomers of strain B, wherein the polypeptide is a single chain and does not form a particle. In some embodiments, the antigenic RSV G polypeptide is a tetramer comprising the amino acids of SEQ ID NO: 5. In some embodiments, the antigenic RSV G polypeptide is a tetramer comprising an amino acid sequence that is 85%, 90%, 95%, or 99% identical to the amino acid sequence of SEQ ID NO: 5.

### 1. RSV G Particles

In some embodiments, an RSV G polypeptide particle or multimer is provided. In some embodiments, an RSV G polypeptide particle or multimer is provided, wherein the particle does not comprise ferritin. In some embodiments, an RSV G polypeptide single chain particle or multimer is provided. In some embodiments, an RSV G polypeptide particle or multimer is provided, comprising any of the RSV G polypeptides described herein and a multimerization domain. In some embodiments, the RSV G particle comprises 1, 2, 3, 4, 5, 6, 7, 8, 9, or 10 multimerization domains. In some embodiments, an RSV G polypeptide comprises 1-2, 1-5, 1-10, 1-20, 1-25, 1-30, 1-40, 1-50, 1-60, 1-70, 1-80, 1-90, 1-100, or 1-120 multimerization domains. In some embodiments, an RSV G polypeptide comprises 3 Gcc multimerization domains. In some embodiments, an RSV G polypeptide comprises 6 Gcc multimerization domains. In some embodiments, an RSV G polypeptide comprises 120 Gcc multimerization domains.

In some embodiments, the RSV G particle comprises 1 multimerization domain. In some embodiments, the multimerization domain is any domain known in the art to self-assemble. In some embodiments, the multimerization domain is a foldon domain. In some embodiments, the foldon domain comprising SEQ ID NO: 11, or a portion of SEQ ID NO: 11 capable of self-assembling. In some embodiments, the foldon domain comprises a sequence that is 80%, 90%, 95%, or 99% identical to SEQ ID NO: 11, wherein the foldon is capable of self-assembling. An exemplary portion of SEQ ID NO: 11 is provided as SEQ ID NO: 13. In some embodiments, the foldon domain comprises a sequence that is 80%, 90%, 95%, or 99% identical to SEQ ID NO: 13, wherein the foldon is capable of self-assembling. In some embodiments, the foldon domain comprises or consists of the sequence of SEQ ID NO: 13.

In some embodiments, the antigenic RSV G polypeptide comprises a sequence having at least 85%, 90%, 95%, 97%, 98%, 99%, or 99.5% identity to amino acids SEQ ID NO: 4 (B1-A2 foldon, with the foldon of SEQ ID NO: 11). In some embodiments, the RSV G polypeptide comprises the sequence of SEQ ID NO: 4. In some embodiments, the antigenic RSV G polypeptide comprises a sequence having at least 85%, 90%, 95%, 97%, 98%, 99%, or 99.5% identity to amino acids SEQ ID NO: 14 (B1-A2 foldon, with the foldon of SEQ ID NO: 13). In some embodiments, the RSV G polypeptide comprises the sequence of SEQ ID NO: 14.

In some embodiments, the antigenic RSV G polypeptide comprises a sequence having at least 85%, 90%, 95%, 97%, 98%, 99%, or 99.5% identity to amino acids SEQ ID NO: 7 (A2-A2 foldon). In some embodiments, the RSV G polypeptide comprises the sequence of SEQ ID NO: 7.

In some embodiments, the antigenic RSV G polypeptide comprises a sequence having at least 85%, 90%, 95%, 97%, 98%, 99%, or 99.5% identity to amino acids SEQ ID NO: 8 (B1-B1 foldon). In some embodiments, the RSV G polypeptide comprises the sequence of SEQ ID NO: 8.

In some embodiments, the RSV G particle is an antigenic RSV G α particle. For example, the RSV G polypeptide may comprise a single chain polypeptide comprising an RSV Gcc from strain A, B, or both A and B, as described herein, and a full or partial α sequence. In some embodiments, the antigenic RSV G polypeptide comprises a sequence having at least 85%, 90%, 95%, 97%, 98%, 99%, or 99.5% identity to SEQ ID NO: 6 (α particle). In some embodiments, the RSV G polypeptide comprises the sequence of SEQ ID NO: 6. In some embodiments, the antigenic α particle is capable of self-assembling. In some embodiments, the antigenic α particle comprises an RSV Gcc and a sequence that is 80%, 90%, 95%, or 99% identical to SEQ ID NO: 9, wherein the particle is capable of self-assembling. In some embodiments, the antigenic α particle displays 60 copies of particle protomer per particle, wherein 2 Gcc's (optionally, one strain A and one strain B) are multimerized so that approximately 120 Gcc's are displayed and available for immune recognition.

In some embodiments, the RSV polypeptide is a single chain construct, e.g., expressed as single polypeptides.

In some embodiments, an RSV Gcc comprises a single amino acid substitution relative to a wild-type sequence. In some embodiments, an RSV Gcc comprises more than one single amino acid substitution, e.g., 2, 3, 4, 5, or 6 substitutions relative to a wild-type sequence. An exemplary wild-type sequence is SEQ ID NO: 3.

### C. Linkers

In some embodiments, a linker separates the amino acid sequence of the RSV monomers and/or multimerization domain, if present. Any linker may be used. In some embodiments, the linker is a peptide linker, which can facilitate expression of the antigenic RSV G polypeptide as a fusion polypeptide (e.g., from a single open reading frame). In some embodiments, the linker is a glycine-serine linker. In some embodiments, the glycine-serine linker is GS, GGGS (SEQ ID NO: 15), 2XGGGS (i.e., GGGSGGGS) (SEQ ID NO: 16), or 5XGGGS (SEQ ID NO: 17).

In some embodiments, the linker is 2, 3, 4, 5, 6, 7, 8, 9, or 10 amino acids in length. In some embodiments, the linker is about 2-4, 2-6, 2-8, 2-10, 2-12, or 2-14 amino acids in length. In some embodiments, the linker is at least 15 amino acids in length. In some embodiments, the linker is at least 25 amino acids in length. In some embodiments, the linker is at least 30 amino acids in length. In some embodiments, the linker is at least 35 amino acids in length. In some embodiments, the linker is at least 40 amino acids in length. In some embodiments, the linker is less than or equal to 60 amino acids in length. In some embodiments, the linker is less than or equal to 50 amino acids in length. In some embodiments, the linker is about 16, 28, 40, 46, or 47 amino acids in length. In some embodiments, the linker is flexible.

In some embodiments, the linker comprises glycine (G) and/or serine (S) amino acids. In some embodiments, the linker comprises or consists of glycine (G), serine (S), asparagine (N), and/or alanine (A) amino acids, and optionally a cysteine as discussed above. In some embodiments, the linker comprises an amino acid sequence with at least 80%, 85%, 90%, 95%, 97%, 98%, 99%, or 100% identity to SEQ ID NO: 222. In some embodiments, the linker comprises GGGGSGGGGSGGGGSG (SEQ ID NO: 220), GGSGSGSNSSASSGASSGGASGGSGGSG (SEQ ID NO: 221), GGSGSASSGASASGSSNGSGSGSGSNSSASSGASSGGASGGSGGSG (SEQ ID NO: 222), or GS. In some embodiments, the linker comprises FR1 (SEQ ID NO: 223) or FR2 (SEQ ID NO: 224).

In some embodiments, a construct does not comprise a linker. In some embodiments, a construct comprises one linker. In some embodiments, a construct comprises two or more than two linkers. In some embodiments, the construct comprises a linker between each monomer and between a monomer and multimerization domain, if present.

### D. Compositions; Uses and Methods for Vaccination

In some embodiments, the invention provides methods of immunizing a subject against infection with RSV. The invention further provides methods of eliciting an immune response against RSV in a subject. In some embodiments, the methods comprise administering to the subject an effective amount of a pharmaceutical composition described herein to a subject. In some embodiments, the methods comprise administering to the subject an effective amount of an antigenic RSV polypeptide, antigenic RSV G particle, or antigenic RSV α particle described herein to a subject.

In some embodiments, a composition comprising any one or more of the polypeptides or particles described herein and a pharmaceutically acceptable vehicle, adjuvant, or excipient is provided.

In some embodiments, a polypeptide, particle, or composition described herein is administered to a subject, such as a human, to immunize against infection caused by RSV. In some embodiments, a polypeptide, nanoparticle, or composition described herein is administered to a subject, such as a human, to produce a protective immune response to future infection with RSV. In some embodiments, any one or more of the polypeptides, nanoparticle, or compositions described herein are provided for use in immunizing against infection caused by RSV. In some embodiments, any one or more of the polypeptides, nanoparticle, or compositions described herein are provided for use in producing a protective immune response to future infection with RSV. In some embodiments, the protective immune response decreases the incidence of infection with RSV, pneumonia, bronchiolitis, or asthma.

In some embodiments, a composition comprises an RSV G polypeptide described herein. In some embodiments, a composition comprises an RSV G particle described herein.

In some embodiments, a composition comprising an RSV G polypeptide described herein elicits a superior neutralizing response to RSV compared to immunization with a post-fusion RSV F polypeptide or Gcc-NP. In some embodiments, immunization with an RSV G polypeptide described herein (e.g., a polypeptide or particle comprising an RSV G polypeptide described herein) elicits a higher titer of antibodies directed against RSV G compared to immunization with Gcc-NP. In some embodiments, immunization with an RSV G polypeptide described herein elicits a higher ratio of total antibody being directed against RSV G compared to immunization with a one or two monomers of Gcc or Gcc-NP. Immunization with an RSV antigen described herein may provide better protection against RSV compared to immunization with a post-fusion RSV F.

In some embodiments, a composition comprising an RSV G polypeptide comprising more than two Gcc monomers, and the Gcc particles described herein elicits a neutralizing response to RSV.

In some embodiments, a composition comprising an RSV G polypeptide comprising more than two Gcc monomers, and the Gcc particles described herein, provide improved protection against RSV, e.g., a higher neutralizing titer than a composition that does not comprise more than two Gcc monomers.

### 1. Subjects

In some embodiments, the subject is a mammal. In some embodiments, the subject is a human.

In some embodiments, the subject is an adult (greater than or equal to 18 years of age). In some embodiments, the subject is a child or adolescent (less than 18 years of age). In some embodiments, the subject is elderly (greater than 60 years of age). In some embodiments, the subject is a non-elderly adult (greater than or equal to 18 years of age and less than or equal to 60 years of age).

In some embodiments, more than one administration of the composition is administered to the subject. In some embodiments, a booster administration improves the immune response.

In some embodiments, any one or more of the antigenic polypeptides, or compositions described herein are for use in a mammal, such as a primate (e.g., non-human primate, such as a monkey (e.g., a macaque, such as rhesus or cynomolgus) or ape), rodent (e.g., mouse or rat), or domesticated mammal (e.g., dog, rabbit, cat, horse, sheep, cow, goat, camel, or donkey). In some embodiments, any one or more of the antigenic polypeptides, or compositions described herein are for use in a bird, such as a fowl (e.g., chicken, turkey, duck, goose, guineafowl, or swan).

### 2. Adjuvants

As described herein, adjuvants may also be administered together with the antigenic RSV G polypeptides and particles described herein to a subject. In some embodiments, administration of adjuvant together with the polypeptide and particles produces a higher titer of antibodies against the RSV polypeptide in the subject as compared to administration of the polypeptide or particle alone, without the adjuvant. An adjuvant may promote earlier, more potent, or more persistent immune response to the antigenic polypeptide.

In some embodiments, a composition comprises one adjuvant. In some embodiments, a composition comprises more than one adjuvant. In some embodiments, a composition does not comprise an adjuvant.

In some embodiments, an adjuvant comprises aluminum. In some embodiments, an adjuvant is aluminum phosphate. In some embodiments, an adjuvant is Alum (Alyhydrogel '85 2%; Brenntag—Cat #21645-51-2).

In some embodiments, an adjuvant is an organic adjuvant. In some embodiments, an adjuvant is an oil-based adjuvant. In some embodiments, an adjuvant comprises an oil-in-water nanoemulsion.

In some embodiments, an adjuvant comprises squalene. In some embodiments, the adjuvant comprising squalene is Ribi (Sigma adjuvant system Cat #S6322-lvl), Addavax™ MF59, AS03, or AF03 (see U.S. Pat. No. 9,703,095). In some embodiments, the adjuvant comprising squalene is a nanoemulsion.

In some embodiments, an adjuvant comprises a polyacrylic acid polymer (PAA). In some embodiments, the adjuvant comprising PAA is SPA09 (see WO 2017218819).

In some embodiments, an adjuvant comprises non-metabolizable oils. In some embodiments, the adjuvant is Incomplete Freund's Adjuvant (IFA).

In some embodiments, an adjuvant comprises non-metabolizable oils and killed Mycobacterium tuberculosis. In some embodiments, the adjuvant is Complete Freund's Adjuvant (CFA).

In some embodiments, an adjuvant is a lipopolysaccharide. In some embodiments, an adjuvant is monophosphoryl A (MPL or MPLA).

### 3. Pharmaceutical Compositions

In various embodiments, a pharmaceutical composition comprising an antigenic RSV G polypeptide described herein is provided. In some embodiments, the pharmaceutical composition is an immunogenic composition (e.g., a vaccine) capable of eliciting an immune response such as a protective immune response against a pathogen.

For example, in some embodiments, the pharmaceutical compositions may comprise one or more of the following: (1) an antigenic RSV G polypeptide comprising more than two RSV Gcc monomers; (2) an antigenic α polypeptide; (3) an antigenic RSV G particle; or (4) an antigenic RSV particle or non-particle trimer, tetramer, or hexamer. In some embodiments, the pharmaceutical composition comprises an antigenic RSV G polypeptide comprising more than two RSV Gcc monomers.

In some embodiments, the present invention provides pharmaceutical compositions comprising antibodies or other agents related to the antigenic polypeptides described herein. In an embodiment, the pharmaceutical composition comprises antibodies that bind to and/or compete with an antigenic polypeptide described herein. Alternatively, the antibodies may recognize viral particles comprising the RSV polypeptide component of an antigenic polypeptide described herein.

In some embodiments, the pharmaceutical compositions as described herein are administered alone or in combination with one or more agents to enhance an immune response, e.g., an adjuvant described above. In some embodiments, a pharmaceutical composition further comprises an adjuvant described above.

In some embodiments, the pharmaceutical composition further comprises a pharmaceutically acceptable carrier or excipient. As used herein, the term “carrier” refers to a diluent, adjuvant, excipient, or vehicle with which a pharmaceutical composition is administered. In exemplary embodiments, carriers can include sterile liquids, such as, for example, water and oils, including oils of petroleum, animal, vegetable, or synthetic origin, such as, for example, peanut oil, soybean oil, mineral oil, sesame oil and the like. In some embodiments, carriers are or include one or more solid components. Pharmaceutically acceptable carriers can also include, but are not limited to, saline, buffered saline, dextrose, glycerol, ethanol, and combinations thereof. As used herein, an excipient is any non-therapeutic agent that may be included in a pharmaceutical composition, for example to provide or contribute to a desired consistency or stabilizing effect. Suitable pharmaceutical excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In various embodiments, the pharmaceutical composition is sterile.

In some embodiments, the pharmaceutical composition contains minor amounts of wetting or emulsifying agents, or pH buffering agents. In some embodiments, the pharmaceutical compositions of may include any of a variety of additives, such as stabilizers, buffers, or preservatives. In addition, auxiliary, stabilizing, thickening, lubricating, and coloring agents can be included.

In various embodiments, the pharmaceutical composition may be formulated to suit any desired mode of administration. For example, the pharmaceutical composition can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. General considerations in the formulation and manufacture of pharmaceutical agents may be found, for example, in Remington's Pharmaceutical Sciences, 19th ed., Mack Publishing Co., Easton, PA, 1995; incorporated herein by reference.

The pharmaceutical composition can be administered via any route of administration. Routes of administration include, for example, oral, intradermal, intramuscular, intraperitoneal, intravenous, subcutaneous, intranasal, mucosal, epidural, sublingual, intranasal, intracerebral, intravaginal, transdermal, rectally, by intratracheal instillation, bronchial instillation, inhalation, or topically. Administration can be local or systemic. In some embodiments, administration is carried out orally. In another embodiment, the administration is by parenteral injection. The mode of administration can be left to the discretion of the practitioner.

In some embodiments, the pharmaceutical composition is suitable for parenteral administration (e.g. intravenous, intramuscular, intraperitoneal, and subcutaneous). Such compositions can be formulated as, for example, solutions, suspensions, dispersions, emulsions, and the like. They may also be manufactured in the form of sterile solid compositions (e.g. lyophilized composition), which can be dissolved or suspended in sterile injectable medium immediately before use. For example, parenteral administration can be achieved by injection. In such embodiments, injectables are prepared in conventional forms, i.e., either as liquid solutions or suspensions, solid forms suitable for solution or suspension in liquid prior to injection, or as emulsions. In some embodiments, injection solutions and suspensions are prepared from sterile powders, lyophilized powders, or granules.

In a further embodiment, the pharmaceutical composition is formulated for delivery by inhalation (e.g., for direct delivery to the lungs and the respiratory system). For example, the composition may take the form of a nasal spray or any other known aerosol formulation. In some embodiments, preparations for inhaled or aerosol delivery comprise a plurality of particles. In some embodiments, such preparations can have a mean particle size of about 1, about 2, about 3, about 4, about 5, about 6, about 7, about 8, about 9, about 10, about 11, about 12, or about 13 microns. In some embodiments, preparations for inhaled or aerosol delivery are formulated as a dry powder. In some embodiments, preparations for inhaled or aerosol delivery are formulated as a wet powder, for example through inclusion of a wetting agent. In some embodiments, the wetting agent is selected from the group consisting of water, saline, or other liquid of physiological pH.

In some embodiments, the pharmaceutical composition in accordance with the invention are administered as drops to the nasal or buccal cavity. In some embodiments, a dose may comprise a plurality of drops (e.g., 1-100, 1-50, 1-20, 1-10, 1-5, etc.).

The present pharmaceutical composition may be administered in any dose appropriate to achieve a desired outcome. In some embodiments, the desired outcome is the induction of a long-lasting adaptive immune response against the source of an RSV polypeptide present in an antigenic particle present in the composition. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

In some embodiments, pharmaceutical compositions in accordance with the invention are administered in single or multiple doses. In some embodiments, the pharmaceutical compositions are administered in multiple doses administered on different days (e.g., prime-boost vaccination strategies). In some embodiments, the pharmaceutical composition is administered as part of a booster regimen.

In various embodiments, the pharmaceutical composition is co-administered with one or more additional therapeutic agents. Co-administration does not require the therapeutic agents to be administered simultaneously, if the timing of their administration is such that the pharmacological activities of the additional therapeutic agent and the active ingredient(s) in the pharmaceutical composition overlap in time, thereby exerting a combined therapeutic effect. In general, each agent will be administered at a dose and on a time schedule determined for that agent.

4. Nucleic acid/mRNA

Also provided is a nucleic acid encoding an antigenic polypeptide or particle described herein. In some embodiments, the nucleic acid is an mRNA. Any nucleic acid capable of undergoing translation resulting in a polypeptide is considered an mRNA for purposes of this disclosure.

### 5. Kits

Also provided herein are kits comprising one or more antigenic polypeptides, nucleic acids, antigenic particles, compositions, or pharmaceutical compositions described herein. In some embodiments, a kit further comprises one or more of a solvent, solution, buffer, instructions, or desiccant.

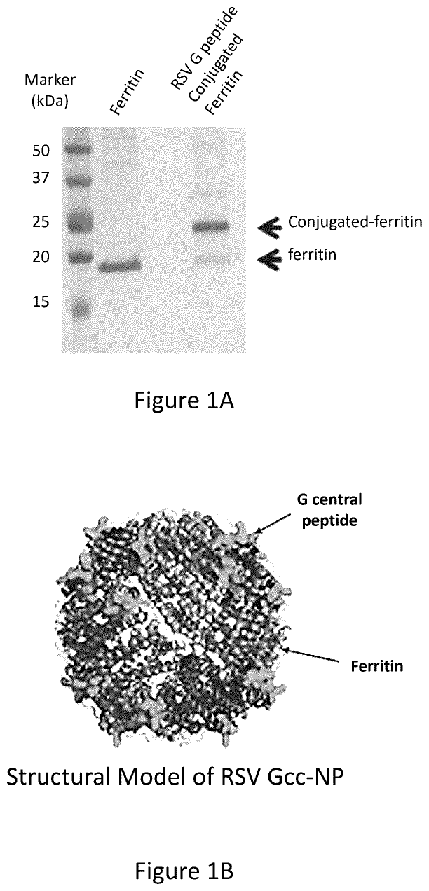

TABLE 1
(Sequence Table): Description of the Sequences

| | | SEQ ID |
|---|---|---|
| Description | Sequence | NO |
| RSV G A strain Native UniProtKB/Swiss-Prot: | MSKNKDQRTAKTLERTWDTLNHLLFISSCLYKLNLKSVAQITLSILAMIISTSLIIVAIIFIASANHKIT | 1 |
| P27022.1 | STTTIIQDATNQIKNTTPTYLTQNPQLGISPSNPSDITSLITTILDSTTPGVKSTLQSTTVGTKNTTTTQ | |
| | AQPNKPTTKQRQNKPPSKPNNDFHFEVFNFVPCSICSNNPTCWAICKRIPNKKPGKRTTTKPTKKPTPKT | |
| | TKKGPKPQTTKSKEAPTTKPTEEPTINTTKTNIITTLLTSNTTRNPELTSQMETFHSTSSEGNPSPSQVS | |
| | ITSEYPSQPSSPPNTPR | |
| RSV G ectodomain, residues 66-297 | NHKVTLTTAIIQDATSQIKNTTPTYLTQDPQLGISFSNLSEITSQTTTILASTTPGVKSNLQPTTVKTKNTTTTQTQPSKPTTKQRQN | 2 |
| | KPPNKPNNDFHFEVFNFVPCSICSNNPTCWAICKRIPNKKPGKKTTTKPTKKPTFKTTKKDHKPQTTKPKEVPTTKPTEEPTINTTKT | |
| | NIITTLLTNNTTGNPKLTSQMETFHSTSSEGNLSPSQVSTTSEHPSQPSSPPNTTRQ | |
| RSV Gcc (central conserved region) A2 | RQNKPPNKPNNDFHFEVFNFVPCSICSNNPTCWAICKRIPNKK | 3 |
| strain res 151-193. As described in Murata et al. | | |
| (2010) Vaccine 28:6242-6246 at FIG. 1. | | |
| RSV Gcc B1-A2-foldon strain chimera sequence | MEESEESGGRKNPPKKPKDDYHFEVFNFVPCSICGNNQLCKSICKTIPNKKEEEEESEESGGrqnkppskpnndfhfevfnfvpcsic | 4 |
| | snnptcwaickripnkkeeessggsggggsggggsggggsgssaiggyipeaprdgqayvrkdgewvllstflgsglevlfqgplehh | |
| | hhhh | |
| RSV Gcc tetramer, A2-B1-A2-B1 strain | MrqnkppskpnndfhfevfnfvpcsicsnnptcwaickripnkRKNPPKKPKDDYHFEVFNFVPCSICGNNQLCKSICKTIPNKKrqn | 5 |
| chimera | kppskpnndfhfevfnfvpcsicsnnptcwaickripnkRKNPPKKPKDDYHFEVFNFVPCSICGNNQLCKSICKTIPNKK | |
| RSV Gcc A2-α-B1 | MEESEESGGrqnkppskpnndfhfevfnfvpcsicsnnptcwaickripnkEEEEESEESGGSGSWEEWNAKWDEWRNDQNDWREDWQ | 6 |
| chimera particle | AWRDDWAYWTLTWRYGELYSRLARIERRVEELRRLLQLIRHENRMVLQFVRALSMQARRLESKLEEEEESEESGGRKNPPKKPKDDYH | |
| | FEVFNFVPCSICGNNQLCKSICKTIPNKKEESEESGG | |
| RSV Gcc A2-A2- | meeseesggrqnkppskpnndfhfevfnfvpcsicsnnptcwaickripnkkEEEEESEESGGrqnkppskpnndfhfevfnfvpcsi | 7 |
| foldon strain | csnnptcwaickripnkkeeessggsggggsggggsggggsgssaiggyipeaprdgqayvrkdgewvllstflgsglevlfqgpleh | |
| chimera sequence | hhhhh | |
| RSV Gcc B1-B1- | MEESEESGGRKNPPKKPKDDYHFEVFNFVPCSICGNNQLCKSICKTIPNKKEEEEESEESGGRKNPPKKPKDDYHFEVFNFVPCSICG | 8 |
| foldon strain | NNQLCKSICKTIPNKKeeessggsggggsggggsggggsgssaiggyipeaprdgqayvrkdgewvllstflgsglevlfqgplehhh | |
| chimera sequence | hhh | |
| α sequence | DEWRNDQNDWREDWQAWRDDWAYWTLTWRYGELYSRLARIERRVEELRRLLQLIRHENRMVLQFVRALSMQARR | 9 |
| RSV Gcc (central conserved region) B1 | RLKNPPKKPKDDYHFEVFNFVPCSICGNNQLCKSICKTIP | 10 |
| strain res 151-193. As described in | | |
| Murata et al. (2010) Vaccine 28:6242-6246 at | | |
| FIG. 1. | | |
| Foldon | aiggyipeaprdgqayvrkdgewvllstflgsglevlfqgple | 11 |
| RSV G peptide A2 (aa 151-193) for conjugation | Azido-PEG4-SGGSSGSSEEEGGSRQNKPPNKPNNDFHFEVFNFVPCSICSNNPTCWAICKRIPNKKEEE | 12 |
| with N-terminal Azido linker and flanking | | |
| glutamates | | |
| 30 amino acid foldon | aiggyipeaprdgqayvrkdgewvllstfl | 13 |
| RSV Gcc B1-A2-30 aa foldon strain chimera | MEESEESGGRKNPPKKPKDDYHFEVFNFVPCSICGNNQLCKSICKTIPNKKEEEEESEESGGrqnkppskpnndfhfevfnfvpcsic | 14 |
| sequence | snnptcwaickripnkkeeessggsggggsggggsggggsgssaiggyipeaprdgqayvrkdgewvllstfl | |
| bfpFerritin-N19Q/C31S/S26C | ESQVRQQFSKDIEKLLNEQVNKEMQSSNLYMCMSSWSYTHSLDGAGLFLFDHAAEEYEHAKKLIIFLNENNVPVQLTSISAPEHKFEG | 201 |
| | LTQIFQKAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/S72C | ESQVRQQFSKDIEKLLNEQVNKEMQSSNLYMSMSSWSYTHSLDGAGLFLFDHAAEEYEHAKKLIIFLNENNVPVQLTCISAPEHKFEG | 202 |
| | LTQIFQKAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/A75C | ESQVRQQFSKDIEKLLNEQVNKEMQSSNLYMSMSSWSYTHSLDGAGLFLFDHAAEEYEHAKKLIIFLNENNVPVQLTSISCPEHKFEG | 203 |
| | LTQIFQKAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/K79C | ESQVRQQFSKDIEKLLNEQVNKEMQSSNLYMSMSSWSYTHSLDGAGLFLFDHAAEEYEHAKKLIIFLNENNVPVQLTSISAPEHCFEG | 204 |
| | LTQIFQKAYEHEQHISESINNIVDHAIKSKDHATFNFLQWYVAEQHEEEVLFKDILDKIELIGNENHGLYLADQYVKGIAKSRKS | |
| bfpFerritin-N19Q/C31S/S100C | ESQVRQQFSKDIEKLLNEQVNKEMQSSNLYMSMSSWSYTHSLDGAGLFLFDHAAEEYEHAKKLIIFLNENNVPVQLTSISAPEHKFEG | 205 |
| | LTQIFQKAYEHEQHISECINNIVDHAIKSKDHATFNFLQWYVAEQHEEEVLFKDILDKIELIGNENHGLYLADQYVKGIAKSRKS | |
| bfpFerritin-N19Q/C31S/S111C | ESQVRQQFSKDIEKLLNEQVNKEMQSSNLYMSMSSWSYTHSLDGAGLFLFDHAAEEYEHAKKLIIFLNENNVPVQLTSISAPEHKFEG | 206 |
| | LTQIFQKAYEHEQHISESINNIVDHAIKCKDHATFNFLQWYVAEQHEEEVLFKDILDKIELIGNENHGLYLADQYVKGIAKSRKS | |
| bfpFerritin-N19Q/C31S/E12C | ESQVRQQFSKDIEKLLNCQVNKEMQSSNLYMSMSSWSYTHSLDGAGLFLFDHAAEEYEHAKKLIIFLNENNVPVQLTSISAPEHKFEG | 207 |
| | LTQIFQKAYEHEQHISESINNIVDHAIKSKDHATFNFLQWYVAEQHEEEVLFKDILDKIELIGNENHGLYLADQYVKGIAKSRKS | |
| Exemplary H. pylori Ferritin with bullfrog linker | ESQVRQQFSKDIEKLLNEQVNKEMNSSNLYMSMSSWCYTHSLDGAGLFLFDHAAEEYEHAKKLIIFLNENNVPVQLTSISAPEHKFEG | 208 |
| | LTQIFQKAYEHEQHISESINNIVDHAIKSKDHATFNFLQWYVAEQHEEEVLFKDILDKIELIGNENHGLYLADQYVKGIAKSRKS | |
| Exemplary wild-type H. pylori ferritin (GenBank | LSKDIIKLLNEQVNKEMNSSNLYMSMSSWCYTHSLDGAGLFLFDHAAEEYEHAKKLIIFLNENNVPVQLTSISAPEHKFEGLTQIFQK | 209 |
| Accession AAD06160.1) (without bullfrog linker | AYEHEQHISESINNIVDHAIKSKDHATFNFLQWYVAEQHEEEVLFKDILDKIELIGNENHGLYLADQYVKGIAKSRKS | |
| or N-terminal Met) | | |
| CpG (ISS-1018) | TGACTGTGAACGTTCGAGATGA | 210 |
| Trichoplusia ni heavy chain ferritin | TQCNVNPVQIPKDWITMHRSCRNSMRQQIQMEVGASLQYLAMGAHFSKDVVNRPGFAQLFFDAASEEREHAMKLIEYLLMRGELTNDV | 211 |
| | SSLLQVRPPTRSSWKGGVEALEHALSMESDVTKSIRNVIKACEDDSEFNDYHLVDYLTGDFLEEQYKGQRDLAGKASTLKKLMDRHEA | |
| | LGEFIFDKKLLGIDV | |
| Trichoplusia ni light chain ferritin | ADTCYNDVALDCGITSNSLALPRCNAVYGEYGSHGNVATELQAYAKLHLERSYDYLLSAAYFNNYQTNRAGFSKLFKKLSDEAWSKTI | 212 |
| | DIIKHVTKRGDKMNFDQHSTMKTERKNYTAENHELEALAKALDTQKELAERAFYIHREATRNSQHLHDPEIAQYLEEEFIEDHAEKIR | |
| | TLAGHTSDLKKFITANNGHDLSLALYVFDEYLQKTV | |
| Pyrococcus furiosus ferritin | MLSERMLKALNDQLNRELYSAYLYFAMAAYFEDLGLEGFANWMKAQAEEEIGHALRFYNY | 213 |
| | IYDRNGRVELDEIPKPPKEWESPLKAFEAAYEHEKFISKSIYELAALAEEEKDYSTRAFL | |
| | EWFINEQVEEEASVKKILDKLKFAKDSPQILFMLDKELSARAPKLPGLLMQGGE | |
| human heavy chain ferritin | MTTASTSQVRQNYHQDSEAAINRQINLELYASYVYLSMSYYFDRDDVALKNFAKYFLHQSHEEREHAEKLMKLQNQRGGRIFLQDIKK | 214 |
| | PDCDDWESGLNAMECALHLEKNVQQSLLELHKLATDKNDPHLCDFIETHYLNEQVKAIKELGDHVTNLRKMGAPESGLAEYLFDKHTL | |
| | GDSDQES | |
| human light chain ferritin (signal peptide | MDSKGSSQKGSRLLLLLVVSNLLLPQGVLASSQIRQNYSTDVEAAVNSLVNLYLQASYTYLSLGFYFDRDDVALEGVSHFFRELAEEK | 215 |
| is underlined) | REGYERLLKMQNQRGGRALFQDIKKPAEDEWGKTPDAMKAAMALEKKLNQALLDLHALGSARTDPHLCDFLETHFLDEEVKLIKKMGD | |
| | HLTNLHRLGGPEAGLGEYLFERLTLKHD | |
| lumazine synthase from Aquifex aeolicus | MQIYEGKLTAEGLRFGIVASRFNHALVDRLVEGAIDCIVRHGGREEDITLVRVPGSWEIPVAAGELARKEDIDAVIAIGVLIRGATPH | 216 |
| | FDYIASEVSKGLANLSLELRKPITFGVITADTLEQAIERAGTKHGNKGWEAALSAIEMANLFKSLR | |
| bullfrog linker | ESQVRQQF | 217 |
| Cysteine-Thrombin-His Linker (cysteine | CLVPRGSLEHHHHHH | 218 |
| is double underlined) | | |
| E. coli 6, 7-dimethyl-8-ribityllumazine | MNIIEANVATPDARVAITIARFNNFINDSLLEGAIDALKRIGQVKDENITVVWVPGAYELPLAAGALAKTGKYDAVIALGTVIRGGTA | 219 |
| synthase | HFEYVAGGASNGLAHVAQDSEIPVAFGVLTTESIEQAIERAGTKAGNKGAEAALTALEMINVLKAIKA | |
| 16 amino acid linker | GGGGSGGGGSGGGGSG | 220 |
| 28 amino acid linker | GGSGSGSNSSASSGASSGGASGGSGGSG | 221 |
| 46 amino acid linker | GGSGSASSGASASGSSNGSGSGSGSNSSASSGASSGGASGGSGGSG | 222 |
| FR1 | GGSGSASAEAAAKEAAAKAGGSGGSG | 223 |
| FR2 | GGSGSASAEAAAKEAAAKEAAAKASGGSGGSG | 224 |
| Not used | | 225 |
| RSV G B strain Native UniProtKB/Swiss-Prot: | MSKHKNQRTARTLEKTWDTLNHLIVISSCLYRLNLKSIAQIALSVLAMIISTSLIIAAIIFIISANHKVT | 226 |
| 036633.1 | LTTVTVQTIKNHTEKNITTYLTQVPPERVSSSKQPTTTSPIHTNSATTSPNTKSETHHTTAQTKGRTTTS | |
| | TQTNKPSTKPRLKNPPKKPKDDYHFEVFNFVPCSICGNNQLCKSICKTIPSNKPKKKPTIKPTNKPTTKT | |
| | TNKRDPKTPAKTTKKETTTNPTKKPTLTTTERDTSTSQSTVLDTTTLEHTIQQQSLHSTTPENTPNSTQT | |
| | PTASEPSTSNSTQNTQSHA | |

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### 1. Preparation of Chimera RSV G Antigens

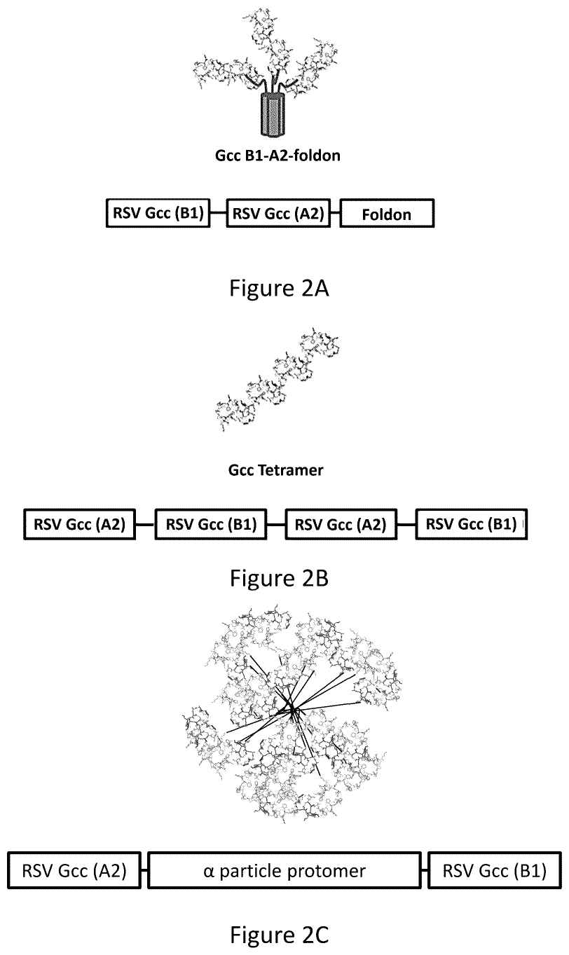

Vectors encoding RSV G chimera polypeptides (i.e., B1-A2-foldon (SEQ ID NO: 4), Gcc tetramer A2-B1-A2-B1 (SEQ ID NO: 5) and A2-α-B1 particle (SEQ ID NO: 6)) were synthesized by Genscript using the pET28 E. coli expression vector, which harbors the IPTG-inducible expression promoter. Modeled structures of the antigens are shown in FIG. 2A-C. The A2-α-B1 particle is expected to comprise 60 protomers, each comprising an A2 Gcc and a B1 Gcc, for ~120 displayed instances of Gcc epitope. Chimeric polypeptides were expressed in E. coli using standard methods in the field. Briefly, BL21 DE3 E. coli cells were transformed with the relevant vector, grown in LB Broth with 0.1% Kanamycin at 37° C. (typically 1 liter expression volumes) and induced with 0.1% IPTG when cell density was around 0.5-0.8 absorbance units (UV 600). Cultures were left overnight (approximately 16 hours) at 18° C. for polypeptide expression. Cells were harvested by centrifugation and the cell pellet was re-suspended in 30 mL PBS and sonicated. Samples were centrifuged 30 mins at 4,000 rpm, supernatant was discarded and retain inclusion bodies and re-suspended in 30 mL of 4 M Urea, followed by sonication. Samples were centrifuged once more and supernatant containing RSV G construct was dialyzed overnight against 4 liters of PBS. Soluble samples were then filtered using 0.22 μm Millex-GP Filter to obtain clear supernatant for polypeptide purification.

HIS-tagged constructs (i.e. B1-A2-foldon (SEQ ID NO: 4)) were initially purified using Ni chelation using GE Healthcare HISTRAP columns, eluting with a gradient of imidazole. Non-HIS-tagged constructs (i.e. Gcc tetramer A2-B1-A2-B1 (SEQ ID NO: 5) and A2-α-B1 particle (SEQ ID NO: 6)) were initially purified using ion exchange GE Healthcare HiTrap Q column with NaCl elution. Fractions containing RSV G constructs were further purified over reverse phase HPLC using a Kinetex 5u C18 100A column to further purify the construct and reduce endotoxin. Finally, RSV G constructs were purified by size exclusion purification using the GE Healthcare Superdex P200 column with Tris Buffered Saline mobile phase. Purity was judged by SDS-PAGE gel and concentration was judged by UV280 absorption, typical in the field.

To demonstrate that the ferritin nanoparticle can be used to improve the immunogenicity of the RSV G central domain antigen we developed a method of chemically conjugating the Gcc peptide (SEQ ID NO: 12) to the ferritin nanoparticle. Ferritin harboring the S111C mutation described herein (SEQ ID NO: 206) can be conjugated with the Gcc peptide (SEQ ID NO: 12) synthesized with a maleimide group on a PEG4 linker attached to the N-terminus via an NHS group. Gcc peptide with an N-terminal maleimide was synthesized and HPLC purified by Peptides International (Louisville, KY, USA). When the maleimide-Gcc antigen is added to the ferritin S111C particle, the maleimide conjugates to the free cysteine and forms a Gcc-NP that can be observed by Coomassie-stained SDS-PAGE gel. While the conjugation is typically 50% to 90% efficient (see FIG. 1A), a model of a Gcc peptide ferritin nanoparticle (100% conjugated) is shown in FIG. 1B.

### 2. In Vivo Characterization of Neutralizing Antibody Responses to RSV G Antigens

To assess the in vivo response to RSV G antigens in mice, female BALBc mice were intramuscularly immunized with RSV antigens at specified doses at week 0, 3 and 6 with either a high dose (5 μg) or low dose (0.5 μg) of antigen. Unless otherwise noted, RSV antigens were adjuvanted with AF03 with a bedside mixing strategy. That is, 50 μl of the relevant polypeptide solution were mixed with 50 μl of Sanofi adjuvant AF03 (a squalene-based emulsion; see Klucker et al., J Pharm Sci. 2012 December; 101(12):4490-500) just prior to injection of 50 μl into each hind leg. No adverse effects from immunization were observed. Blood was collected 1 day prior to first immunization and at least 2 weeks after each injection (i.e. weeks 2, 5 and 8). Unless otherwise specified, data shown was for 2 weeks post third injection (week 8, also denoted as 2wp3). Typically, sera were analyzed from pre-immunized animals (denoted as naïve), two weeks post second injection (post-2 or 2wp2) or two weeks post third injection (post-3^{rd} or 2wp3).

For the HAE neutralizing assay, serum was heat-inactivated for 30 minutes at 56° C. A fourfold serial dilution series of the inactivated serum was made in PneumaCult™-ALI Basal Medium (Stem Cell Technologies; 05002) supplemented with PneumaCult™-ALI 10× Supplement (Stem Cell Technologies; 05003) and 1% Antibiotic/Antimycotic (hence media). RSV viral stocks were combined 1:1 with the serum dilutions and incubated for 1.5 hours at 37° C. The virus-serum mixture was then added to 24 well plates containing fully differentiated HAE cells at 50 μL per well and incubated for 1 hour at 37° C., 5% CO₂. Following incubation, the inoculum was removed, the wells were washed twice with media to remove unbound virus and incubated a further 20 hours at 37° C., 5% CO₂. Infection events in cultures infected with RSV expressing the mKate (TagFP635) reporter were counted on a fluorescent microscope.

To detect infection with RSV not expressing the mKate reporter (RSV B strain neutralization), the pseudostratified epithelia were washed extensively with media to remove mucus then fixed with 4% paraformaldehyde for 30 minutes at room temperature, permeabilized with 0.25% Triton X-100 for 30 minutes, and blocked with DMEM supplemented with 2% FBS for 1 hour at 37° C. The blocking solution was replaced with 100 μL per well of Mouse Anti-RSV monoclonal Ab mixture (Millipore; MAB 858-4) diluted 1:200 in DMEM supplemented with 2% FBS, and the plates were incubated at 37° C. for 2 hours. The plates were then washed 3 times with PBS supplemented with 0.05% Tween 20. 100 μL of Goat anti-mouse IgG (H+L) (Invitrogen; A11001) diluted 1:200 in DMEM supplemented with 2% FBS was added per well, and the plates were incubated overnight at 4° C. Next morning, the plates were washed 3 times with PBS supplemented with 0.05% Tween 20, the florescent signal was stabilized with ProLong Gold AntiFade with DAPI (Thermo Fisher Scientific; P36935) and counted on a fluorescent microscope. The neutralizing antibody titers were determined at the 60% reduction end-point.

### 3. In Vivo Characterization of Binding Antibody Responses to RSV G Antigens

For anti-Gcc binding, a trimerized dimer of Gcc peptide with a C-terminal HIS tag was used on an Octet tip similar to above. His₆-tagged Gcc (A2 strain) hexamer (SEQ ID NO: 7) or His₆-tagged Gcc (B1 strain) hexamer (SEQ ID NO: 8) was pre-loaded onto Anti-Penta-HIS (HIS1K) sensor tips (FortéBio #18-5122) for 400 seconds to allow capture to reach near saturation. Biosensor tips were then equilibrated for 90 seconds in Octet Wash Buffer, followed by diluted sera association for 300 seconds. Association curve final responses were measured using Octet Data Analysis HT10.0 software, and the response was multiplied by the dilution factor (100 or 300) to obtain the final reported response.

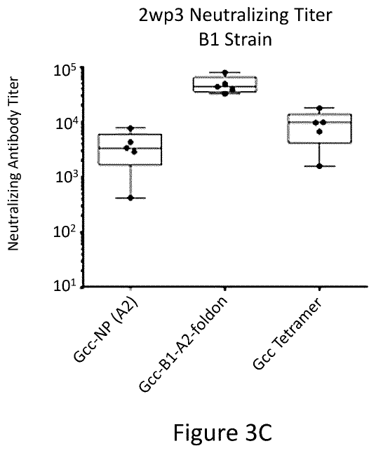

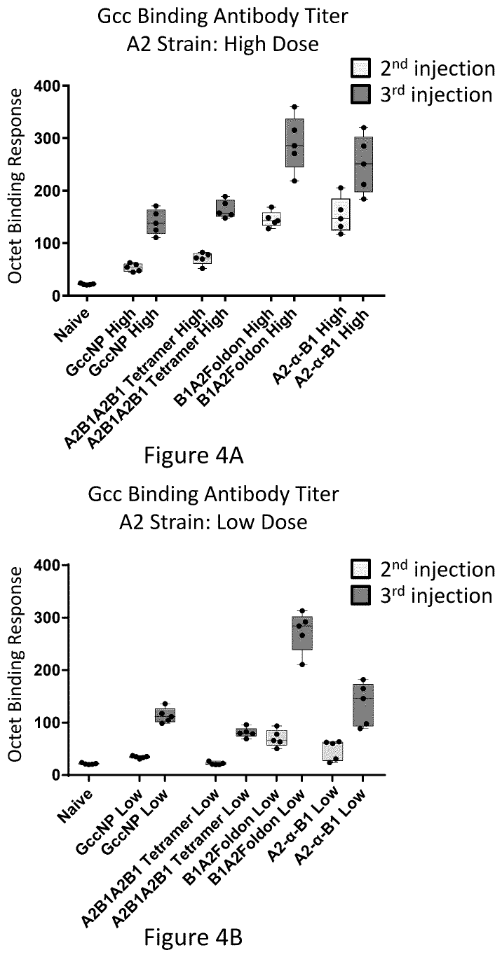

To determine if the RSV G antigens elicit a Gcc-binding immune response the sera from the immunizations above were tested for their ability to bind Gcc A2 hexamer (SEQ ID NO: 7) or Gcc B1 hexamer (SEQ ID NO: 8). The Gcc-binding response at high dose (FIG. 4A and FIG. 5A) and low dose (FIG. 4B and FIG. 5B) were tested at 2 weeks post-second and 2 weeks post-third immunizations. For both A2 strain (FIGS. 4A-B) and B1 strain (FIG. 5A-B), all antigens elicited a binding response relative to naïve mice sera. At all timepoints and doses, B1-A2-foldon elicited a superior binding response to Gcc A strain relative to Gcc tetramer or Gcc-NP. At each timepoint, high dose A2-α-B1 elicited binding response similar to high dose B1-A2-foldon, but at low dose A2-α-B1 elicited a reduced Gcc A strain-binding response relative to low dose B1-A2-foldon (FIG. 4A-B).

At high dose at all timepoints, B1-A2-foldon, A1B2A1B2 tetramer and A2-α-B1 elicited a superior binding response to Gcc B strain relative to high dose Gcc-NP (FIG. 5A). At each timepoint, low dose A2-α-B1 and B1-A2-foldon elicited Gcc B1-binding superior to low dose Gcc-NP and A1B2A1B2 tetramer (FIG. 5B).

### 4. Response in Human Cells

To demonstrate the ability of the Pre-F-NP antigen and the Gcc-NP antigen to elicit a response in human cells, experiments are performed with the MIMIC platform. The MIMIC platform is comprised solely of autologous human immune cells capable of quickly and reproducibly generating antigen-specific innate and adaptive responses upon challenge. Previous work has demonstrated the ability of the MIMIC system to recapitulate in vivo immune profiles against such diverse targets as HBV, tetanus toxoid, monoclonal antibodies, YF-VAX, and influenza B-cell responses. To demonstrate Gcc-NP elicits a superior G antibody response than Gcc peptide alone, human cells are treated with Gcc peptide alone or Gcc peptide conjugated to nanoparticle (Gcc-NP) in human B-cells. Gcc-NP elicits a superior G-binding antibody response. Thus, it is expected that particles comprising Gcc epitopes will elicit immune responses in human immunization.

### 5. Characterization of Antibody Binding to Gcc Antigen

Antibody binding to a chimeric Gcc-foldon polypeptide having the sequence of SEQ ID NO: 14 was evaluated using a double sandwich ELISA (Enzyme Linked Immuno Sorbent Assay).

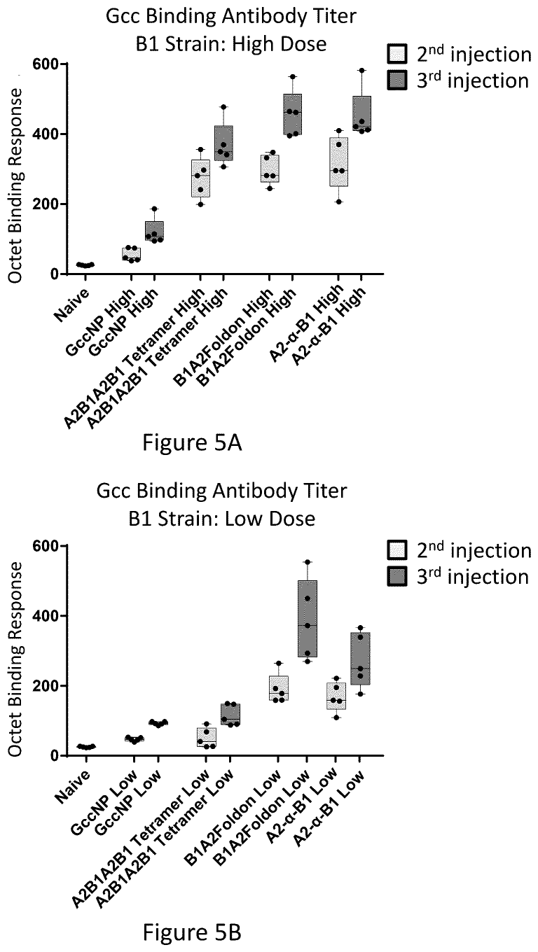

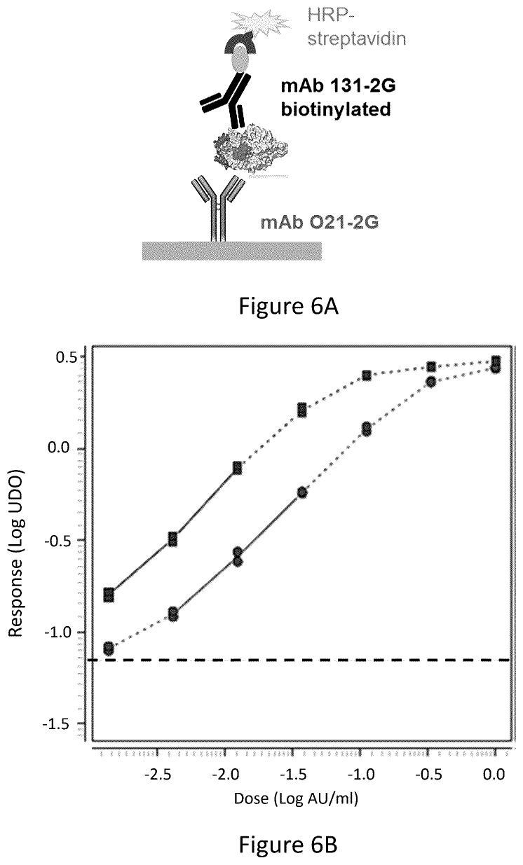

In the assay, RSV G glycoprotein was trapped between the capture antibody and the detection antibody. In this ELISA the detection antibody was labeled with biotin and the sandwich detected using enzyme-conjugated streptavidin (FIG. 6A).

To study whether the Gcc polypeptide had the proper conformation, the 021-2G Mab antibody (humanized purified anti-protein G monoclonal antibody, clone [021-2G], supplier RD Biotech) was used, which recognizes a conformational epitope of Gcc. Thus, 021-2G Mab binding indicates that the Gcc polypeptide is not misfolded or degraded. The detection antibody was purified anti-protein G monoclonal antibody (Mouse IgG1, kappa), clone [131-2G], Supplier: Sigma, ref: MAB 858-2-5, biotinylated using a biotinylation kit such as Lynx rapid plus biotin (type1) antibody conjugation kit biotin (type1) (Biorad ref LNK263B, LNK262B, or LNK261B) or Biotin EZ link sulfo NHS-LC-Biotin (Thermo Scientific ref 21327).

The results are shown in FIG. 6B, indicating that the chimeric Gcc-foldon polypeptide having the sequence of SEQ ID NO: 14 was recognized by both anti-protein G antibodies used, including clone [021-2G], which recognizes a conformational epitope of Gcc. This evidence is consistent with the conclusion that the chimeric Gcc-foldon polypeptide has a proper conformation for eliciting antibodies that will recognize native RSV.

### Methods

To perform the assay, the following steps were performed:

Prepare a 021-2G coating antibody solution at 1 μg/ml in PBS 1×. Distribute 100 μL per well throughout a 96-well plate. Cover with plate sealer.

Incubate the sealed plate 16 h to 20 h at +5° C.±3° C. May be stored for up to 3 months at ≤−70° C. When the plates are thawed: drain and then proceed directly to saturation.

Defrost and empty plates. Distribute 200 μL/well of saturation buffer (1×PBS with 0.05% polysorbate 20 and 1% milk) throughout the plate. Cover with plate sealer. Place the plate in a plate incubator. Incubate at least 1 hour at approximately +37° C.

Perform 3 washes of the plate in Wash Buffer (1×PBS with 0.05% polysorbate 20 (Sigma P1379)).

All dilutions are realized with dilution buffer (iX PBS with 0.05% polysorbate 20 and 0.1% milk).

Foldon Gcc-His tag (non soluble fraction), supplier: BTL, was used as the reference antigen and internal control. Dilutions of the reference antigen and the internal control were made in independent duplicates from the same aliquot. The dilutions prepared for the reference antigen and the internal control were made in glass “Hemolysis” type test tube or in 4.5 mL plastic NUNC cryotubes.

Prepare a 2-fold dilution series of the reference, internal controls, and sample, each at 100 μL volume in a 96-well plate. Also prepare two blank wells containing dilution buffer only. Seal the plate with plate sealer. Place the plate in a plate incubator. Incubate for approximately 1 hour at approximately +37° C. Perform 3 washes in Wash Buffer.

Prepare a detection antibody solution at the appropriate concentration in dilution buffer. Distribute 100 μL/well of the detection antibody solution throughout the plate. Seal the plate with a plate sealer. Place the plate in a plate incubator. Incubate for approximately 1 hour at approximately +37° C. Perform 3 washes in Wash Buffer.

Prepare a solution of rabbit polyclonal anti-human IgG antibody coupled to peroxidase at the appropriate dilution buffer concentration. Distribute 100 μL/well of the conjugate solution throughout the plate. Seal the plate with a plate sealer. Place the plate in a plate incubator. Incubate for approximately 1 hour at approximately +37° C. Perform 3 washes in Wash Buffer.

Distribute 100 μL/well of TMB (ready-to-use 3,3′,5,5′-Tetramethylbenzidine peroxidase substrate solution) throughout the plate. Incubate for approximately 10 min at room temperature and in the dark (e.g., wrapped in foil). Stop the reaction by adding 100 μL/well of 1N HCl solution. Read plates with a plate reader at 450 and 620 nm. Measure optical density (OD) expressed as the difference between the 2 readings to take into account the absorption of the plastic of the 96-well plate.

The calculation of relative activity is carried out by the method of parallel lines in an application respecting the formulas of the European Pharmacopoeia (paragraph 5.3.3: parallel line assays) and qualified by the ARD EU: PLA 2.0 (Parallel-Line Assays) or equivalent distributed by the company STEGMANN. The Log/Log model is used to calculate the relative activity in accordance with dose response law modeling.

1. An antigenic RSV G polypeptide comprising more than two RSV G central conserved region (Gcc) monomers and a multimerization domain, wherein the polypeptide does not comprise ferritin and/or the polypeptide is a single chain polypeptide,
wherein the more than two RSV Gcc monomers comprise RSV Gcc monomers of strain A or strain B,
wherein the RSV Gcc of strain A comprises:
a) the same sequence of amino acids as amino acids 2-42 of SEO ID NO: 4, or
b) the same sequence of amino acids as amino acids 151-193 of SEO ID NO: 1;
wherein the RSV Gcc of strain B comprises the same sequence of amino acids as amino acids 10-51 of SEO ID NO: 8; and
wherein the multimerization domain comprises the sequence of SEO ID NOs: 9, 11, or 13.

2. (canceled)

3. (canceled)

4. The antigenic RSV G polypeptide of claim 1, comprising 3, 4, 5, 6, 7, 8, 9, or 10 Gcc monomers.

5. (canceled)

6. The antigenic RSV G polypeptide of claim 1, comprising more than two Gcc monomers of strain A.

7. The antigenic RSV G polypeptide of claim 1, comprising more than two Gcc monomers of strain B.

8. (canceled)

9. The antigenic RSV G polypeptide of claim 1, wherein the polypeptide is a trimer, tetramer, or hexamer.

10. The antigenic RSV G polypeptide of claim 1, comprising three Gcc monomers of strain A.

11. The antigenic RSV G polypeptide of claim 1, comprising three Gcc monomers of strain B.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The antigenic RSV G polypeptide of claim 1, capable of forming a Gcc particle upon expression of the polynucleotide in a host cell.

29. (canceled)

30. (canceled)

31. The antigenic RSV G polypeptide of claim 1, wherein the antigenic RSV G polypeptide is encoded by a polynucleotide encoding at least three RSV Gcc monomers.

32. The antigenic RSV G polypeptide of claim 31, wherein the polynucleotide encodes four RSV Gcc monomers.

33. (canceled)

34. (canceled)

35. The antigenic RSV G polypeptide of claim 1, wherein the antigenic RSV G polypeptide is capable of eliciting an immune response to RSV and/or protecting a subject against RSV infection.

36. A composition comprising the antigenic RSV G polypeptide of claim 1, further comprising a pharmaceutically acceptable carrier.

37. The antigenic RSV G polypeptide or composition of claim 1 for use in a method of eliciting an immune response to RSV or in protecting a subject against RSV infection.

38. A method of eliciting an immune response to RSV or protecting a subject against RSV infection comprising administering the antigenic RSV G polypeptide or composition of any one of claim 1 to a subject.

39. The antigenic RSV G polypeptide or composition of claim 36, wherein the subject is human.

40. A nucleic acid encoding the antigenic RSV polypeptide of claim 1, optionally wherein the nucleic acid is an mRNA.

41. The method of claim 38, wherein the subject is human.

42. The antigenic RSV G polypeptide of claim 1, comprising an amino acid sequence with 80%, 85%, 90%, 95%, 98%, or 99% identity to any one of SEQ ID NOs: 4, 6, or 14.

43. The antigenic RSV G polypeptide of claim 1, comprising the amino acid sequence of any one of SEQ ID NOs: 4, 6, or 14.
